(19) United States
(12) Patent Application Publication
(10) Pub. No.: US 2016/0109472 A1
(43) Pub. Date: Apr. 21, 2016

(54) QUANTITATIVE MOLAR CONCENTRATION DETECTION OF SPECIFIC APOLIPOPROTEIN-CONTAINING PARTICLES PRESENT IN BODILY FLUIDS BY USING CAPILLARY ELECTROPHORESIS

(71) Applicant: HEALTH DIAGNOSTIC LABORATORY, INC., Richmond, VA (US)

(72) Inventors: Philip GUADAGNO, Mechanicsville, VA (US); Erin Grace Summers BELLIN, Sandston, VA (US); Deepika DEVANUR, Richmond, VA (US); Stuart HASSARD, Richmond, VA (US)

(21) Appl. No.: 14/919,464

(22) Filed: Oct. 21, 2015

Related U.S. Application Data
(60) Provisional application No. 62/066,573, filed on Oct. 21, 2014.
(60) Provisional application No. 62/147,665, filed on Apr. 15, 2015.

(51) Int. Cl. G01N 33/92; G01N 27/447; G01N 21/64

(52) U.S. Cl. CPC G01N 33/92; G01N 21/6428; G01N 27/447; G01N 2800/32; G01N 2800/50; G01N 2021/6439

(57) ABSTRACT

A method for determining the molar concentration of specific lipoprotein particles present in a bodily fluid is presented. Multipixel Capillary Isotachophoresis Laser Induced Fluorescence is applied to fluorescently-labeled lipoproteins or immunologically-labeled apolipoproteins, facilitating quantification of lipoproteins and/or lipid particles and/or their associated apolipoproteins in a sample. The measurements are used to predict the risks of developing, progressing in severity of diseases related to lipoprotein particles, including cardiovascular and metabolic disorders.

This application claims the benefit of U.S. Provisional Patent Application Ser. No. 62/066,573, filed Oct. 21, 2014 and U.S. Provisional Patent Application Ser. No. 62/147,665 filed Apr. 15, 2015, which are hereby incorporated by reference in their entirety.

### FIELD OF THE INVENTION

The present invention is relates to a method and a system for determining the molar concentration and/or particle number of a lipoprotein present in a biological sample. The invention also teaches a method for assessing a cardiovascular risk in a subject.

### BACKGROUND OF THE INVENTION

Lipoproteins are biological assemblies comprising an outer layer of protein and phospholipids and a core of neutral lipids including cholesterol esters and triacylglycerols. Lipoproteins include very low-density lipoprotein (VLDL), low-density lipoprotein (LDL), intermediate-density lipoprotein (IDL), high-density lipoprotein (HDL), chylomicrons, and lipoprotein(a) (Lp(a)) particles. Each lipoprotein particle is further divided into subclasses, which vary in size, density, protein, and lipid composition. It is well-established that the lipoprotein subclass distribution profile of an individual may be indicative of a health risk. In particular, cardiovascular and metabolic disorders are correlated strongly with specific patterns of subclass quantity and size (see U.S. Pat. No. 6,518,069).

Apolipoproteins are structural components of lipoprotein particles and are bound to water-insoluble lipid molecules by covalent or non-covalent forces in a specific stoichiometry (see U.S. patent application Ser. No. 14/194,142). Apolipoprotein species include, but are not limited to, apolipoprotein A (apoA), apolipoprotein B (apoB), apolipoprotein C (apoC), apolipoprotein D (apoD), apolipoprotein E (apoE), apolipoprotein H (apoH), and apolipoprotein (a). U.S. patent application Ser. No. 14/194,142 describes the association of apolipoprotein particles with specific lipoproteins.

Various disease states, including but not limited to cardiovascular disease, liver disease, and diabetes mellitus, are associated with the levels of apolipoproteins and/or lipoprotein particles (see, e.g., U.S. Pat. No. 6,518,064). For example, apoB is a constituent of VLDL and LDL particles, which are associated with increased risk of cardiovascular disease. Increased levels of Lp(a), which comprise an LDL-like particle with apoA bound to apoB by a disulfide bond, is associated with an increased risk of early atherosclerosis independent of other cardiac risk factors. Moreover, differences in the amount of cholesterol in a particle may also correlate with the risk of cardiovascular disease. For example, elevated levels of small, dense, cholesterol ester rich LDL correlate with an increased risk of cardiovascular disease; while elevated levels of cholesterol rich HDL correlate with a decreased in risk of cardiovascular disease. Thus, the risk of developing a cardiovascular disease can be assessed by quantifying the levels of these lipoproteins.

Several methods have been developed to separate and characterize lipoprotein classes and subclasses in a biological sample, including nuclear magnetic resonance spectroscopy (NMR), ultracentrifugation, and electrophoresis. Analysis using NMR spectroscopy involves analyzing a chemical shift spectrum for lipoprotein subclasses. While NMR can be used to detect HDL, VLDL, IDL and LDL, it cannot be used to detect Lp(a) particles. Likewise, the applicability of NMR to lipoprotein characterization is limited by the low sample throughput and cost of equipment. Lp(a) can be measured using specialized techniques like immuno-fixation and acid violet staining. However, such techniques are labor intensive and not automatable.

Direct quantification of lipoprotein cholesterol can be achieved by enzymatic assay of the individual lipoproteins, which can be separated by electrophoresis or selective precipitation. The most accurate method for isolating lipoprotein is ultracentrifugation; but, this method is very time consuming and expensive and therefore not suitable for large-scale population studies.

Electrophoresis is the separation of charged molecular species based on the size and/or ionic charge of the molecules in an electric field. Biological molecules, including proteins, amino acids, and nucleic acids, possess ionizable groups that allow them to migrate as charged particles in an electric field. When separation occurs in a capillary, the method is known as capillary electrophoresis (CE).

CE encompasses a family of related separation techniques that use narrow-bore fused-silica capillaries to separate a complex array of large and small molecules. High electric field strengths are used to separate molecules based on differences in charge, size and hydrophobicity. Sample introduction is accomplished by immersing the end of the capillary into a sample vial and applying pressure, vacuum or voltage. Depending on the types of capillary and electrolytes used, the technology of CE can be segmented into several separation techniques. Exemplary CE techniques include isoelectric focusing, isotachophoresis (ITP), and capillary zone electrophoresis, also known as free-solution capillary electrophoresis. Separation of lipoproteins by capillary electrophoresis is an effective technique for accurately detecting the lipid particles and relative subfractions. These methods are limited by the absence of effective and scalable methods to calculate lipid particle concentration.

CE-ITP is an electrophoretic technique in which sample ions are separated under an electric field across a length of tubing or capillary. A liquid plug comprising a biological sample to be separated is bounded by a leading buffer on one end and a trailing buffer on the other end. The leading and trailing buffers maintain the sample between them, enhancing the separations resolution. As samples migrate through the capillary, the sample components focus into bands based on their unique electrophoretic mobilities. Such bands can be distinguished by various techniques including UV light absorption, native fluorescence directly in the capillary or after elution from the capillary by subsequent gel or immunological detection.

CE-ITP has been used to separate plasma lipoproteins in preparation for subsequent analysis on a gradient gel (see Bottcher et al., “Automated Free-Solution Isotachophoresis: Instrumentation and Fractionation of Human Serum Proteins,” Electrophoresis. 19(7): 1110-6 (1998) and Bottcher et al., “Preparative Free-Solution Isotachophoresis for Separation of Human Plasma Lipoproteins: Apolipoprotein and Lipid Composition of HDL Subfractions,” J Lipid Res. 41(6): 905-15 (2000)). In particular, Bottcher describes that sample components were separated from one another through the use of spacers. Analysis required use of a transfer gel, gradient gel electrophoresis and western blotting for detection. This and other current capillary isotachophoresis methods do not permit the quantification of the molar quantities of lipoproteins present in a biological sample, which is a more accurate predictor of the levels of lipoprotein subparticles, and the risk of developing a disease.

No study to date has incorporated particle separation and detection using a single instrument in an automated format. Additional substrates, transfer steps, and detection modes in an assay severely limit this type of application. Moreover, limitations on the detectable analytes fail to offer sufficient information for health assessment. Resolution in the existing methods fails to offer insight in lipid particle subclass or variant characterization. Thus, there is great variability among the available separation methods in terms of accuracy, convenience, and cost. Additionally, current methods including ultracentrifugation or NMR do not provide molar concentrations of apoB containing lipid particles like Lp(a).

It is desirable to characterize lipid particles from patients in a single experimental protocol, or with a single substrate supporting separation and detection; to provide detailed resolution of particle types in a separation protocol; to quantify said particles and particle subclasses in the abbreviated experimental protocol; and to quantify constant molar mass apolipoproteins on separated lipid particles with known and constant stoichiometry for quantifying molar concentrations of apolipoproteins per particle. This invention is directed to overcoming these constraints in the art.

### SUMMARY OF THE INVENTION

A first aspect of the invention relates to a method for determining the molar concentration and/or particle number of a lipoprotein in a biological sample. The method involves contacting a biological sample with a fluorophore-labeled antibody under conditions suitable for the fluorophore-labeled antibody to bind to a lipoprotein, or an immunologically active component thereof, to form a fluorophore-labeled lipoprotein. The method further involves subjecting the fluorophore-labeled lipoprotein to a capillary isotachophoresis laser-induced fluorescence (CE-ITP-LIF) system; detecting signals produced by the fluorophore-labeled lipoprotein; and quantifying, based on said detecting, the molar concentration and/or particle number of the lipoprotein in the sample, wherein the detected signals are proportional to the molar concentration and/or particle number of the lipoprotein in the sample.

A second aspect of the invention relates to a method of assessing cardiovascular risk in a subject. This method involves determining the particle number and/or molar concentration of a lipoprotein in a biological sample. The method further involves assessing the cardiovascular risk of the subject based on the particle number and/or molar concentration of the lipoprotein, where the particle number and/or molar concentration of the lipoprotein is determined by (a) contacting a biological sample with a fluorophore-labeled antibody under conditions suitable for the fluorophore-labeled antibody to bind to the lipoprotein, or an immunologically active component thereof, to form a fluorophore-labeled lipoprotein; (b) subjecting the fluorophore-labeled lipoprotein to a capillary isotachophoresis laser-induced fluorescence (CE-ITP-LIF) system; (c) detecting signals produced by the fluorophore-labeled lipoprotein; and (d) quantifying, based on said detecting, the molar concentration and/or particle number of the lipoprotein in the sample, wherein the detected signals are proportional to the molar concentration and/or particle number of the lipoprotein in the sample.

A third aspect of the invention relates to a system for determining the molar concentration and/or particle number of a lipoprotein in a biological sample. This system comprises a capillary electrophoresis apparatus for separating components of a moiety-bound sample, wherein the moiety-bound sample is prepared by contacting the biological sample with a fluorophore-labeled antibody under conditions suitable for the fluorophore-labeled antibody to bind to the lipoprotein or an immunologically active component thereof, to form a fluorophore-labeled lipoprotein. The system also comprises a detector for detecting signals produced by the fluorophore-labeled lipoprotein and a processor for quantifying, based on said detecting, the concentration and/or particle number of the lipoprotein in the sample, where the detected signals are proportional to the molar concentration and/or particle number of the lipoprotein in the sample.

The methods and system described herein provide for the simultaneous separation and detection of lipoprotein particles present in a biological sample. The advantage of the methods and system of the present invention include the fast, accurate, and detailed resolution of lipoprotein particle classes and subclasses, as well as the ability to quantify the particle number and/or molar concentration of multiple lipoprotein particles, including Lp(a), in a biological sample.

### BRIEF DESCRIPTION OF THE DRAWINGS

FIG. 1 is a schematic drawing of a system comprising two optics zones. Optics zone 1 comprises an optical rail on which are arranged a 445 nm or other specific wavelength laser or laser diode. Light form these sources is focused through a series of optical components comprising, but not limited to, a line generator, a crossed linear polarizer, and a neutral density filter. Light from Optics zone 1 is focused onto a 12.5 mm area of a 100 μM internal diameter fused silica capillary (˜365 μM o.d.) in which a 20 mm viewing window has been created by the thermal removal of the polyamide sheath. The light then passes through the sample that is being separated by ITP and excites the fluorescent label attached to each analyte molecule. Emitted light energy, at a wavelength specific to the fluorescent label is then focused to a 512 pixel photo diode array (“PDA”) through another series of optical component called Optics zone 2. Optics zone 2 comprises a set of imaging lenses (e.g., convex lenses), and an orthogonal crossed linear polarizer. After passing through a cut-on filter that transmits above a certain wavelength, the light energy reaches the detector where the data is acquired on the PDA and the signal is processed by proprietary signal processing algorithms.

FIG. 2 is a schematic drawing of a system comprising two optics zones. Optics zone 1 comprises a 445 nm LED/Laser/Laser Diode. Optics zone 2 comprises an off axis concave diffusion grating that focuses wavelength dispersed achromatic light of a wavelength specific to the fluorescent label onto the 512 pixel photo diode array. By rotating the diffraction grating, the light energy reaches the detector where the data is acquired on the PDA and the signal is processed by proprietary signal processing algorithms. An additional cut-on filter or crossed polarizer may be added.

FIG. 3 is a schematic drawing of a system comprising a simple off axis translucent parabolic mirror.

FIG. 4 is a schematic drawing of a system comprising two optics zones. Optics zone 2 comprises a fibre-optic plate (“FOP”) or coherent fibre bundle allowing proximity focusing via a cut-on filter without needing the PDA to touch the capillary.

FIGS. 5A-5C show the analysis of collected pixel data. FIG. 5A is a typical electropherogram from a single pixel of the PDA used to build up the Equiphase map shown in FIG. 5B. Each point of the Equiphase map represents a detected peak in space (pixel) and time (scan count). Tracking is performed to group sets of peaks into signal tracks, which travel in a straight line across the Equiphase map. FIG. 5C shows the fitting of such tracks with linear functions to give their velocities. Each black line in FIG. 5C represents a signal track; the gradient of the lines gives the velocity.

FIGS. 6A-6C are electropherograms of multiple samples showing the detection of individual fractions by CE-ITP-ILF. FIG. 6A shows an electropherogram of a control sample comprising CF in the absence of a biological sample. FIG. 6B shows the lipoprotein profile of several replicate biological samples prepared from patient 8 and spiked with CF. FIG. 6C shows that the lipid profile detected remains constant even after CF has degraded.

FIGS. 7A-7C are electropherograms of native samples from patient 8 prepared in the presence or absence of a lipoprotein spike. FIG. 7A shows an electropherogram of a native sample of patient 8 incubated with an HDL spike. FIG. 7B shows an electropherogram of a native sample of patient 8 incubated with an LDL spike. FIG. 7C shows an electropherogram of a HDL/VDL/LDL mixture from patient 8 incubated with a VLDL spike. The arrow indicated the possible location of the VDL peak.

FIGS. 8A-8E are electropherograms showing the lipid profiles of various biological samples. FIG. 8A shows an alignment of electropherograms from samples prepared from LDL Patient 6 (top) and LDL Patient 4 (bottom). Gel images (not shown) indicate that the LDL 6 sample contains Lp(a) and that the LDL 4 sample does not. FIG. 8B shows the alignment of electropherograms from samples prepared from patients 1-6. Samples from Patients 1, 2, and 6 should contain Lp(a). Arrows indicate possible extra peaks which could indicate the presence of Lp(a). FIG. 8C shows 3 replicate electropherograms of the HDL sample from patient 6. FIG. 8D shows the alignment and normalization of electropherograms from HDL samples of patients 1-6. Electropherograms were normalized around the CF peak (arrow). FIG. 8E shows the alignment and reproducibility triplicate electropherograms from native samples.

FIGS. 9A-9G show the lipid profiles of 6 biological samples. FIGS. 9A-9F show electropherograms of biological samples from patients 1-6, respectively. FIG. 9G shows an alignment of the electropherograms collected for biological samples from patients 1-6, normalized around the CF peak. Corrected peak areas are shown in the figure. Arrows indicate peaks corresponding to CF and LDL peaks.

### DETAILED DESCRIPTION

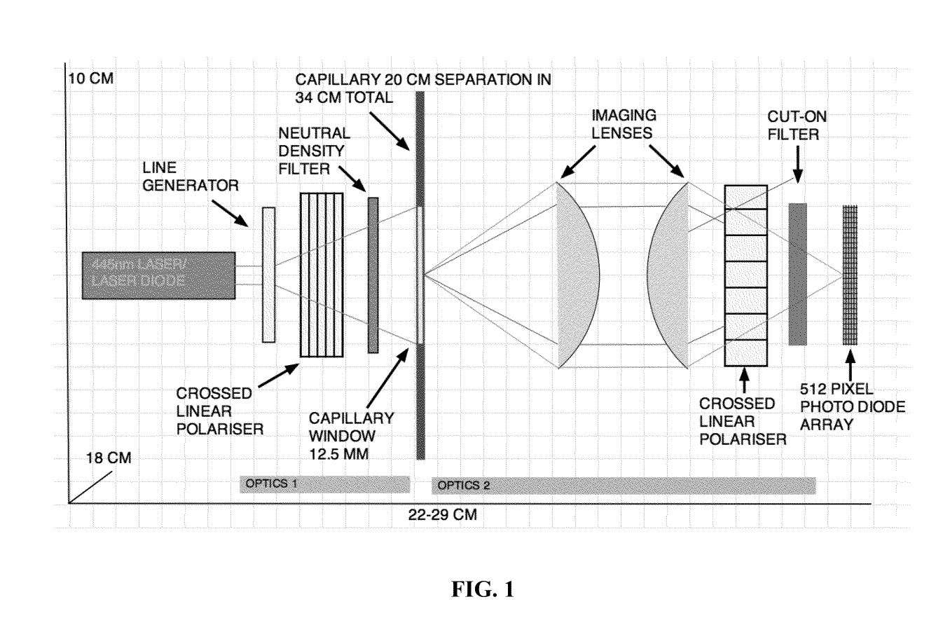

The present invention is directed to a method and a system for determining the molar concentration and/or particle number of a lipoprotein present in a biological sample. The invention also teaches a method for assessing a health risk in a subject. These methods use a CE-ITP-LIF apparatus, which provides for the simultaneous resolution and detection of labeled lipoproteins or lipid particles present in a biological sample (see FIGS. 1-4 and Example 1).

One aspect of the invention relates to a method for determining the molar concentration and/or particle number of a lipoprotein in a biological sample. The method involves contacting a biological sample with a fluorophore-labeled antibody under conditions suitable for the fluorophore-labeled antibody to bind to a lipoprotein, or an immunologically active component thereof, to form a fluorophore-labeled lipoprotein. The method further involves subjecting the fluorophore-labeled lipoprotein to a capillary isotachophoresis laser-induced fluorescence (CE-ITP-LIF) system; detecting signals produced by the fluorophore-labeled lipoprotein; and quantifying, based on said detecting, the molar concentration and/or particle number of the lipoprotein in the sample, wherein the detected signals are proportional to the molar concentration and/or particle number of the lipoprotein in the sample.

The term “lipoprotein particle” refers to a particle that contains both protein and lipid. Examples of lipoprotein particles are described in more detail below.

The terms “lipoprotein particle number” or “molar concentration” as used herein refer to the number of lipoprotein particles present in a unit volume of a biological sample. The lixpoprotein particle number (PN) may be in units of nmol/L.

The term “apolipoprotein” as used herein refers to a protein that combines with lipids to form a lipoprotein particle. Examples of apolipoprotein types are described in more detail below. The unique nature of the apolipoprotein is their stoichiometric relationship to lipoprotein particles, providing an estimate of the lipoprotein particle number, which is described in more detail below.

Suitable biological samples according to the invention include, without limitation, fresh blood, stored blood, or blood fractions. The sample may be a blood sample expressly obtained for the assays of this invention or a blood sample obtained for another purpose which can be subsampled for use in accordance with the methods according to the invention. For instance, the biological sample may be whole blood. Whole blood may be obtained from the subject using standard clinical procedures. The biological sample may also be plasma. Plasma may be obtained from whole blood samples by centrifugation of anti-coagulated blood. The biological sample may also be serum.

Additional exemplary biological samples include, without limitation, human biological matrices, urine, plasma, serum, blood components, synovial fluid, ascitic fluid, and human lipoprotein fractions. The lipid fraction may be substantially pure such that it comprises a single lipoprotein class or subclass. Alternatively, the lipid fraction may be unpurified and comprise one or more lipoprotein particle classes or subclasses.

As described in more detail herein, apolipoproteins are the protein component of lipoprotein particles. Apolipoproteins coat lipoprotein particles that include cholesterol esters and triacylglyceride. The coat of the lipoprotein particle is made up of unesterified cholesterol, phospholipids, and apolipoproteins. The unique nature of the apolipoprotein is their stoichiometric relationship to lipoprotein particles, providing an estimate of the lipoprotein particle number. For example, apolipoprotein B is found on IDL, LDL, VLDL and Lp(a) in 1:1 stoichiometric ratios. Likewise, detection with an anti-apoB antibody detects a composite of at least LDL and Lp(a) particles.

According to the methods of the present invention, biological samples comprise a lipoprotein, or an immunologically active component thereof. In one embodiment, the lipoprotein is an apolipoprotein-containing lipoprotein selected from the group consisting of very-low density lipoprotein (VLDL), low-density lipoprotein (LDL), intermediate-density lipoprotein (IDL), high-density lipoprotein (HDL), chylomicron, lipoprotein X, lipoprotein(a), and subforms and mixtures thereof. In accordance with this embodiment, the apolipoprotein is selected from the group consisting of apolipoprotein A, apolipoprotein B, apolipoprotein C, apolipoprotein D, apolipoprotein E, apolipoprotein H, and oxidized variants and mixtures thereof.

Additional exemplary immunologically active components include, but are not limited to, carbohydrate moieties and apolipoprotein subclass specific antigens. Apolipoprotein subclasses are known in the art and include apoA-I, apoA-II, and apoA-IV. An immunologically active component may target an apoA-I subclass specific antigen.

In one embodiment, the immunologically active component of the lipoprotein comprises an apolipoprotein or domain thereof.

Immunologically active components are contacted with a fluorophore-labeled antibody to form a fluorophore-labeled lipoprotein. In one embodiment, the fluorophore-labeled antibody is an intact antibody or antibody fragment. An exemplary antibody fragment is a fragment antigen-binding fragment.

Fluorophores may be used to form fluorophore-labeled antibody using well-known techniques in the art.

Many suitable fluorophores are known in the art (see, e.g., “The Handbook—A Guide to Fluorescent Probes and Labeling Technologies,” Molecular Probes, Inc., Eugene, Oreg., (2004), which is hereby incorporated by reference in its entirety). Such dyes are known by those skilled in the art and may be chosen from a group including, but not limited to, Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, Cy®3, Cy®5, Fluorescein (FITC), Oregon Green®, Pacific Blue™, Pacific Green™, Pacific Orange™, Tetramethylrhodamine (TRITC), Texas Red®, and Texas Red®.

In some embodiments, the contacting step involves one or more fluorophore-labeled antibodies. The contacting step may involve one fluorophore-labeled antibody. Alternatively, the contacting step may involve two fluorophore-labeled antibodies. The contacting step may also involve three, four, five, six, seven, eight, or nine antibodies.

In other embodiments, a biological sample is contacted with a fluorophore-labeled antibody cocktail which comprises two or more fluorophore-labeled antibodies. In accordance with these embodiments, the antibody cocktail comprises a first fluorophore-labeled antibody targeting a first immunologically active component and a second fluorophore-labeled antibody targeting a second immunologically active component, where the fluorescent emission spectra of the first fluorophore-labeled antibody does not significantly overlap with the fluorescent emission spectra of the second fluorophore-labeled antibody. Likewise, when a fluorophore-labeled antibody cocktail comprises three, four, five, six, seven, eight, or nine fluorophore-labeled antibodies directed to different immunologically active components, the fluorescent emission spectra of each fluorophore-labeled antibody do not significantly overlap with each other. Antibody cocktails and fluorescent detection are described in more detail in U.S. Patent Application Publication No. 2014/0243431, which is hereby incorporated by reference in its entirety.

In one embodiment, an antibody cocktail is directed to two different apolipoprotein classes or subclasses. Alternatively, the antibody cocktail may be directed to three, four, five, six, seven, eight, or nine apolipoprotein different apolipoprotein classes or subclasses. Exemplary apolipoprotein subclasses are described in U.S. patent application Ser. No. 14/194,142, which is hereby incorporated by reference in its entirety.

In an example, the first antigen-specific epitope is directed to an apoB particle and the second antigen-specific epitope directed to an apoA particle. Likewise, a first antibody may target the apoB lipoprotein found on LDL, VLDL, and Lp(a) particles; and a second antibody may target apoA apolipoproteins found on Lp(a) particles.

The methods of the present invention utilize a capillary isotachophoresis laser induced fluorescence (CE-ITP-LIF) system to simultaneously separate and detect the signal produced by labeled lipoproteins in a biological sample. Labeled lipoprotein particles are separated into a spectrum of bands comprising similar molecules.

In one embodiment, the CE-ITP-LIF system separates the components of the sample from one another along a common capillary.

In another embodiment, the CE-ITP-LIF system is a multiplex capillary isotachophoresis laser induced fluorescence (MPCE-ITP-LIF) system. MPCE-ITP-LIF systems comprise a parallel array of capillaries to simultaneously analyze multiple biological samples.

The CE-ITP-LIF and/or MPCE-ITP-LIF systems use a light source or a laser beam with an appropriate emission band to excite a fluorophore-labeled lipoprotein sample. As the fluorophore-labeled lipoprotein components of the biological sample pass through the detection window of the system, the fluorophore is excited by a laser beam of the appropriate wavelength to induce a signal (i.e., a characteristic fluorescent emission maximum).

In one embodiment, the CE-ITP-LIF and/or MPCE-ITP-LIF systems use one laser beam with an appropriate emission band to excite one fluorophore or non-specific dye. Alternatively, the CE-ITP-LIF and/or MPCE-ITP-LIF systems may use one laser beam with an appropriate emission band to excite one or more fluorophores. Likewise, the CE-ITP-LIF and/or MPCE-ITP-LIF system may use more than one laser beam with the appropriate emission bands to excite two, three, four, five, six, seven, eight, nine, or any number of fluorophores.

In each of the preceding embodiments, the CE-ITP-LIF and/or MPCE-ITP-LIF system may be equipped with a detector to enable detection of the signal produced by the fluorophore-labeled lipoprotein and/or signal-producing calibrator lipoprotein.

In one embodiment, the detector is a multipixel detector. An exemplary multipixel detector is a photodiode array.

Systems with CE-ITP capability are known in the art. An exemplary CE-ITP system is made by deltaDOT Ltd. Such instruments may require several modifications to perform the methods of the present invention. For example, in order to use an Alexa Fluor® 488 fluorophore, which is preferentially excited at 488 nm wavelength, in the method of the present invention, a CE-ITP system may be modified comprise a laser with a specific wavelength (e.g., 445 nm, 473 nm, or 488 nm) to illuminate the capillary for the measurement of fluorophore levels migrating past the observation window. Additionally, the system may be modified to comprise a series of optical components (e.g., lenses and filters) in front of the detector (e.g, a photodiode array), to focus the light beam and narrow the wavelength absorbed to that expected from the Alexa-488 fluorophore. Exemplary optical systems of the present invention are described in FIGS. 1-4 and Example 1.

In accordance with this embodiment of the invention, the labelled-lipoprotein is excited by a light source and emits a signal which is detected by a photodiode array, which detects signals over time and space. A computer in communication with the instrument collects the signal emission data and converts it to a form interpretable by a person, such as an electropherogram generated by signal processing algorithms, or a form for further computational analysis.

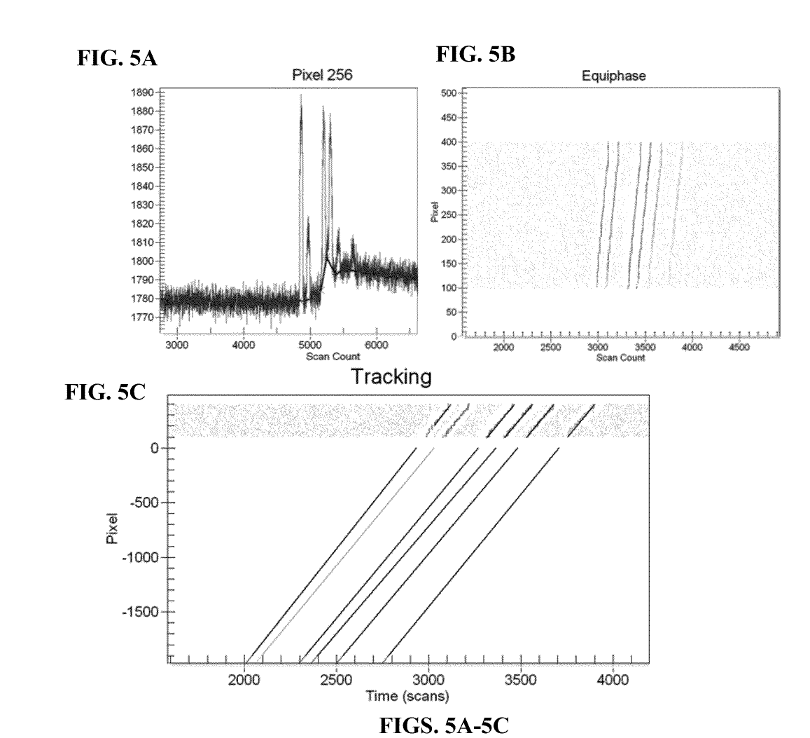

As described herein, an electropherogram is a plot of results recording the separated components of a biological sample produced by capillary electrophoresis (see FIG. 5 and Examples 2-5). The electropherogram may comprise several peaks, each corresponding to the relative molar concentration and/or particle number of a fluorophore-labeled lipoprotein component in the biological sample (see Examples 2-5). The total area under each peak corresponds to the total signal detected in a sample.

If the total concentration of an apolipoprotein component in a sample is known, then the relative concentration of the apolipoprotein corresponding to each lipoprotein peak can be calculated. In any of the preceding embodiments, the signal produced by the fluorophore-labeled antibody is measured and the molar concentration and/or particle number of the lipoprotein is determined based on the following formula:

PN=(relative AUC value)×(total apolipoprotein concentration);

where PN is equal to particle number in

$$( \frac{nmol}{L} ) ;$$

(relative AUC value) is determined by calculating the relative optical density of a specific peak as a portion of the optical density of all peaks; and (total apolipoprotein concentration) is given in

$$( \frac{nmol}{L} )$$

and is determined by measuring the specific lipoprotein concentration.

The relative AUC value may be determined by calculating the relative area under the curve of a specific peak as a portion of the area under the curve of all peaks in an electropherogram.

As indicated above, the molar concentration and/or particle number of a lipoprotein class or subclass in a biological sample can be quantified based on detecting signals produced by a fluorophore-labeled lipoprotein. The detected signals are proportional to the molar concentration and/or particle number of the fluorophore labeled-lipoprotein in the sample.

In any of the preceding embodiments, the sample further comprises a signal-producing calibrator lipoprotein comprising a standard lipoprotein with a known concentration, a known particle number, a known apolipoprotein domain, or a combination thereof.

In one embodiment, the signal produced by the signal-producing calibrator lipoprotein is measured and compared with the signal produced from the fluorophore-labeled lipoprotein and the molar concentration and/or particle number of the lipoprotein is determined based on said comparison.

In another embodiment, the signals produced from the fluorophore-labeled antibody bound to the lipoprotein, or to the immunologically active component thereof, are proportional to the molar concentration and/or particle number of the lipoprotein in the sample.

A biological sample may comprise several lipoprotein class or subclass particles, each comprising the same apolipoprotein. For example, apoA is a component of HDL, chylomicron, and Lp(a) particles; apoB is a component of VLDL, IDL, LDL, and Lp(a) particles; apoC is a component of chylomicron, VLDL, IDL, and HDL particles; apoD is a component of HDL particles; and apoE is a component of chylomicron and IDL particles (see, e.g., U.S. patent application Ser. No. 14/194,142, which is hereby incorporated by reference in its entirety).

In an example, a fluorophore-labeled apoB antibody is used to label the lipoprotein components of a biological sample comprising VLDL, IDL, LDL, and Lp(a) particles. During the subjecting step, the sample is separated into bands corresponding to each fluorophore-labeled lipoprotein component. In particular, the signal detected is proportional to the molar concentration and/or particle number of the apoB component of each detected lipoprotein class (see Examples 2-5). Since apoB is found on VLDL, IDL, LDL, and Lp(a) in a 1:1 stoichiometry, the molar concentration and/or particle number of apoB corresponds to the molar concentration and/or particle number of VLDL in the VLDL band, IDL in the IDL band, LDL in the LDL band, and Lp(a) in the Lp(a) band. Thus, an advantage of the methods of the present invention includes the ability simultaneously separate and quantifies each of the fluorophore-labeled lipoprotein particle components of a biological sample.

In any of the preceding embodiments, a non-specific dye is infused in the lipid portions of lipoproteins including Lp(a), HDL, IDL, LDL, VLDL, and mixtures, composites, subclasses, or superclasses thereof. In accordance with this embodiment, the identities of the labeled-lipoprotein particles are determined by their characteristic position within the migrating sample (see Examples 2-5).

According to the methods of the present invention, biological samples and/or lipoprotein standards may be contacted with a non-specific lipophilic dye under conditions suitable for the non-specific lipid dye to bind to the lipoprotein or lipid particle thereof, to form a lipophilic dye-labeled lipoprotein. Suitable non-specific lipophilic dyes include fluorescently-tagged lipid anchors (e.g., fluorescently-labeled fatty acid analogs). Such optically-active components may be broadly termed lipophilic dyes, with or without the lipid anchor. An example of labeled fatty acid analog is NDB-ceramide. The NDB moiety is a useful label in the hydrophobic environment of a lipid membrane, as it has drastically different optical properties than its properties in an aqueous environment outside the lipid particle and lipid membrane. Other possible fluorescent label-linked fatty acids include ADIFAB fatty acid indicators, phospholipids with BODIPY dye-labeled acyl chains such as BODIPY glycerophospholipids, phospholipid with DPH-labeled acyl chain, phospholipids with NBD-labeled acyl chains, phospholipids with pyrene-labeled acyl chains, phospholipids with a fluorescent or biotinylated head group, LipidTOX phospholipid and neutral lipid stains. Many such options are provided by Life Technologies™ for research and production laboratory assays.

Other exemplary non-specific lipophilic dyes include, without limitation, carboxyfluorescein, BODIPY dyes, or the Alexa Fluor™ series. Such dyes are known by those skilled in the art and may be chosen from a group including, but not limited to lipophilic versions of fluorescent dyes including Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, BODIPY® FL, Coumarin, Cy®3, Cy®5, Fluorescein (FITC), Oregon Green®, Pacific Blue™, Pacific Green™, Pacific Orange™, Tetramethylrhodamine (TRITC), Texas Red®, DNA stains, DAPI, Propidium Iodide, SYTO® 9, SYTOX® Green, TO-PRO®-3, Qdot® probes, Qdot® 525, Qdot® 565, Qdot® 605, Qdot® 655, Qdot® 705, Qdot® 800, other lipophilic fluorescein derivatives such as carboxyfluorescein, carbocyanine derivatives such as iD (DiIC18[5]), DiI (or DiIC18[3]), DiI in vegetable oil, Dilinoleyl DiI, Dilinoleyl DiO, DiO (or DiOC18[3]), DiOC14(3), hydroxyethanesulfonate, DiOC16(3), DiR (DiIC18[7]), DiSC2(5), DODC (DiOC2(5)), Neuro-DiI, Neuro-DiI in vegetable oil, Neuro-DiO, Neuro-DiO in vegetable oil.

When using a lipid anchor, a variety of options may be chosen from the group including, but not limited to fatty acids, phospholipids, acyl chains such as glycerophospholipids, and neutral lipids.

Contacting the lipophilic dyes with an uncharacterized biological sample comprising lipoprotein and/or lipid particles is done to saturation of the lipid particle membranes. In addition to time, mixing and heating and cooling steps may facilitate rapid saturation of the label in the membrane.

A second aspect of the invention relates to a method of assessing cardiovascular risk in a subject. This method involves determining the particle number and/or molar concentration of a lipoprotein in a biological sample. The method further involves assessing the cardiovascular risk of the subject based on the particle number and/or molar concentration of the lipoprotein, where the particle number and/or molar concentration of the lipoprotein is determined according to the first aspect of the invention.

In one embodiment, the subject is a mammal selected from the group including, but not limited to, a human, a non-human primate, a rodent, a canine, a feline, and a bovied.

In another embodiment, the subject is a human.

The subject may be healthy. Alternatively, the subject may be known to suffer from a cardiovascular or metabolic disorder and/or at risk of suffering from a cardiovascular or metabolic disorder. The subject may be a patient suspected of suffering from a lipoprotein-associated disorder including, but not limited to, cardiovascular disorders and obesity. Additional lipoprotein disorders include hyperlipidemia (i.e., the abnormal elevation of lipids or lipoproteins in the blood), arteriovascular disease, atherosclerosis, pancreatitis, and liver disorders. Moreover, elevated or unbalanced lipid and lipoprotein levels are reflective of a subject's development of or progression of diabetic conditions and metabolic disorders.

As described above, suitable biological samples according to the invention include, without limitation, fresh blood, stored blood, or blood fractions.

The method involves (a) contacting a biological sample with a fluorophore-labeled antibody under conditions suitable for the fluorophore-labeled antibody to bind to a lipoprotein, or an immunologically active component thereof, to form a fluorophore-labeled lipoprotein; (b) subjecting the fluorophore-labeled lipoprotein to a capillary isotachophoresis laser-induced fluorescence (CE-ITP-LIF) system; (c) detecting signals produced by the fluorophore-labeled lipoprotein; and (d) quantifying, based on said detecting, the molar concentration and/or particle number of the lipoprotein in the sample, wherein the detected signals are proportional to the molar concentration and/or particle number of the lipoprotein in the sample.

In one embodiment, the CE-ITP-LIF system separates the components of the sample from one another along a common capillary. In another embodiment, the CE-ITP-LIF system is a multiplex capillary isotachophoresis laser induced fluorescence (MPCE-ITP-LIF) system. In accordance with this embodiment, the MPCE-ITP-LIF system separates multiple samples simultaneously.

In each of the preceding embodiments, the CE-ITP-LIF and/or MPCE-ITP-LIF system may be equipped with an appropriate detection device to enable detection of the signal produced by the fluorophore-labeled lipoprotein and/or signal-producing calibrator lipoprotein.

In one embodiment, the detector is a multipixel detector. An exemplary multipixel detector is a photodiode array.

In another embodiment, the sample further comprises a signal-producing calibrator lipoprotein comprising a standard lipoprotein with a known concentration, a known particle number, a known apolipoprotein domain, or a combination thereof. In accordance with this embodiment of the invention, the signal produced by the signal-producing calibrator lipoprotein is measured and compared with the signal produced from the fluorophore-labeled lipoprotein and the molar concentration and/or particle number of the lipoprotein is determined based on said comparison.

The concentration of a labeled-lipoprotein class or subclass may be calculated by comparing the signal produced by a biological sample to the signal produced by a signal-producing calibrator lipoprotein. In one embodiment, the signals produced from the fluorophore-labeled antibody bound to the lipoprotein, or to the immunologically active component thereof, are proportional to the molar concentration and/or particle number of the lipoprotein in the sample.

This aspect of the invention involves assessing the cardiovascular risk of the subject based on the particle number and/or molar concentration of the lipoprotein in a biological sample from a subject.

Lipoprotein particle profiles are different for different individuals and for the same individual at different times. The lipoprotein particles or portions thereof to be assessed for determining a health risk include, but are not limited to, VLDL, LDL, IDL, HDL, chylomicron, lipoprotein X, Lp(a), and subforms and mixtures thereof.

Chylomicrons are produced in the intestine and transport digested fat to the tissues. Lipoprotein lipase hydrolyzes triacylgylcerol to form fatty acids. Chylomicrons are one of the largest buoyant particles. VLDL is formed from free fatty acids upon metabolism of chylomicrons in the liver. Lipoprotein lipase hydrolyzes triacylgylcerol to form fatty acids. IDL is the unhydrolyzed triacylglycerol of VLDL. IDL becomes LDL due to hepatic lipase. HDL plays a role in the transfer of cholesterol to the liver from peripheral tissue. HDL is synthesized in the liver and intestines.

LDL particles bind to LDL receptors. Upon receptor binding, LDL is removed from the blood. Cells use cholesterol within the LDL for membranes and hormone synthesis. LDL deposits LDL cholesterol on the arterial wall which contributes to cardiovascular disease. LDL causes inflammation when it builds up inside an artery wall. Macrophages are attracted to the inflammation and turn into foam cells when they take up LDL, causing further inflammation. Smaller, denser LDL contain more cholesterol ester than the larger, buoyant LDL.

The structure of the LP(a) is that of an LDL-like particle with apolipoprotein A bound to apolipoprotein B by a disulfide bond. Lp(a) particles appear to play a role in coagulation and may stimulate immune cells to deposit cholesterol on arterial walls. A high Lp(a) level indicates a higher risk for cardiovascular disease. Therefore, Lp(a) is useful in diagnostic and statistical risk assessment. Lp(a) may serve to facilitate LDL plaque deposition. Levels of Lp(a) are increased in atherogenic events.

Lp(a) may have a link between thrombosis and atherosclerosis, interfering with plasminogen function in the fibrinolytic cascade. Numerous studies have documented the relationship of high plasma Lp(a) concentrations to a variety of cardiovascular disorders, including peripheral vascular disease, cerebrovascular disease, and premature coronary disease. One large study of older Americans, in particular, demonstrated elevated levels of Lp(a) independently predict an increased risk of stroke, death from vascular disease, and death from all causes in men (see Fried et al., “The Cardiovascular Health Study: Design and Rationale,” Ann. Epidemiol. 3:263-76 (1991), which is hereby incorporated by reference in its entirety).

In one embodiment of the methods of the present invention, the particle number and/or molar concentration of a lipoprotein in a biological sample is used to determine the lipoprotein distribution of the biological sample. The lipoprotein distribution may comprise the relative amounts of each lipoprotein particle in a biological sample. The lipoprotein distribution may also state the particle number and/or molar concentration of a lipoprotein particle in a biological sample.

Risk for development or progression of cardiovascular disease or metabolic disorders like diabetes may be determined by the comparison of measured values of ApoB, Lp(a), lipoproteins, lipid particles, and combinations or ratios of those values to the value from a population distribution. Alternatively, the values may be used in some risk or index score calculation. The risk level is reported to a patient or his or her health care provider for treatment considerations.

In one embodiment, the subject is assigned to one of a low, moderate, or high cardiovascular risk categories based on the particle number and/or molar concentration of the lipoprotein.

There are well established recommendations for cut-off values for biochemical markers (for example, and without limitation, lipoprotein levels) for determining a health risk. For instance, the cut-off values for assigning such risk categories may be as follows: Lp(a): <75 nmol/L optimal, 76-125 nmol/L intermediate risk, >126 nmol/L high risk; LDL: <1000 nmol/L optimal, 1000-1299 nmol/L intermediate risk, >1300 nmol/L high risk.

In accordance with this embodiment, the method further comprises administering to the subject a therapeutic regimen for reducing cardiovascular risk, or modifying an existing therapeutic regimen for the subject for reducing the cardiovascular risk, based on the cardiovascular risk category assigned to the subject.

In an embodiment, the therapeutic regimen comprises administering a drug and/or a supplement or modifying an existing therapeutic regimen by administering a modified dose of a drug and/or a supplement.

The drug or supplement may be any suitable drug or supplement useful for the treatment or prevention of a cardiovascular disease.

In some embodiments, the drug is selected from the group consisting of niacin, an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, an angiotensin system inhibitor, and combinations thereof. Alternatively, the drug is selected from the group consisting of niacin, statin, ezetimibe, fenofibrate, estrogen, raloxifene and combinations thereof.

The agent may be administered in an amount effective to treat the cardiovascular disorder, metabolic disorder, diabetes, or any combination thereof or to lower the risk of the subject for developing a future cardiovascular disorder, metabolic disorder, diabetes, or any combination thereof.

In some embodiments, the selected therapeutic regimen involves giving recommendations on making or maintaining lifestyle choices based on the results of said health risk determination. In accordance with this embodiment, the lifestyle choices involve changes in diet, changes in exercise, reducing or eliminating smoking, or a combination thereof.

In any of the preceding embodiments according to this aspect of the invention, the biological sample is selected from the group consisting of blood, plasma, urine and saliva.

In each of the preceding embodiments, the signal produced by the fluorophore-labeled antibody is measured and the molar concentration and/or particle number of the lipoprotein is determined based on the following formula:

PN=(relative AUC value)×(total apolipoprotein concentration);

where PN is equal to particle number in

$$( \frac{nmol}{L} ) ;$$

(relative AUC value) is determined by calculating the relative optical density of a specific peak as a portion of the optical density of all peaks; and (total apolipoprotein concentration) is given in

$$( \frac{nmol}{L} )$$

and is determined by measuring the specific lipoprotein concentration.

The relative AUC value may be determined by calculating the relative area under the curve of a specific peak as a portion of the area under the curve of all peaks in an electropherogram.

The method may further involve calculating a score of measured levels of apolipoprotein by converting the apolipoprotein concentration to molar lipid concentration; comparing the score to a score obtained from a population of patients; and assigning a cardiovascular risk level to the subject, based on said comparing.

In accordance with this embodiment of the present invention, the total apolipoprotein concentration was measured by polyacrylamide gel electrophoresis. The use of polyacrylamide gel electrophoresis for detecting apolipoprotein concentration is well known in the art (see, e.g., Vezina et al., “Apolipoprotein Distribution in Human Lipoproteins Separated by Polyacrylamide Gradient Gel Electrophoresis,” J Lipid Res. 29(5): 573-85 (1988), which is hereby incorporated by reference in its entirety).

The method may further comprise reporting the risk level to the patient or patient's health care provider.

A third aspect of the invention relates to a system for determining the molar concentration and/or particle number of a lipoprotein in a biological sample. This system comprises a capillary electrophoresis apparatus for separating components of a moiety-bound sample, wherein the moiety-bound sample is prepared by contacting the biological sample with a fluorophore-labeled antibody under conditions suitable for the fluorophore-labeled antibody to bind to the lipoprotein or an immunologically active component thereof, to form a fluorophore-labeled lipoprotein. The system also comprises a detector for detecting signals produced by the fluorophore-labeled lipoprotein and a processor for quantifying, based on said detecting, the concentration and/or particle number of the lipoprotein in the sample, where the detected signals are proportional to the molar concentration and/or particle number of the lipoprotein in the sample.

The system comprises a separation apparatus to isolate lipoprotein particles and lipoprotein subclasses in the bodily fluid based on their ionic mobilities using a capillary electrophoresis (CE-ITP) apparatus and a detector for detecting signals indicating the presence of labeled-lipoprotein particles. In one embodiment, the system is a capillary isotachophoresis laser-induced fluorescence (CE-ITP-LIF) system. In accordance with this embodiment, the system is a multiplex capillary isotachophoresis laser induced fluorescence (MPCE-ITP-LIF) system. MPCE-ITP-LIF systems are described in detail above, in FIGS. 1-4, and Example 1 of the present application. In accordance with this embodiment, the MPCE-ITP-LIF system separates multiple samples simultaneously.

The CE-ITP and or MCPE-ITP system further comprises a laser-induced fluorescence (LIF) detector for detecting a signal emitted from the fluorescent dye or a fluorophore label. The signal is used to quantitate the level of said specific lipoprotein particles.

In one embodiment, the system is a CE-ITP-LIF system. In another embodiment, the system is an MCPE-LIF system.

As noted in the accompanying Figures (FIGS. 1-4) and Example 1, the apparatus may include a laser and set of optical components such as lenses and filters. Lasers may be used to excite the labelled-apolipoprotein or lipoprotein particle. Filters may be used to limit light hitting the detectors by intensity, focal length and wavelength, so that only the fluorophore of interest is monitored.

In an example, an Alexa Flour® 488 fluorophore may be used to label and detect a specific apolipoprotein or lipoprotein particle. Alternatively, carboxyfluorescein may be used to detect the phospholipids of a lipoprotein particle. In order to measure the signal produced by an Alexa Flour® 488 labeled apolipoprotein or lipoprotein particle, the system may be equipped with a 488 nm laser. Alternatively, the system may be equipped with a 408 nm laser in order to detect a carboxyfluorescein labeled lipoprotein particle.

In one embodiment, the separation apparatus is flanked by two optical zones. The first optical zone may comprise a specific wavelength laser. The second optical zone may comprise a detector to execute laser-induced fluorescence measurements.

The detector detects the signal produced by a labeled apolipoprotein or lipoprotein particles. In one embodiment, the detector is a multipixel detector. An exemplary multipixel detector is a photodiode array.

The system also comprises a processor connected to the detector to process the detected fluorescent signal into an output value for interpretation by another processor or a human. In one embodiment, the processor is programmed with signal processing algorithms to process the signal by (i) reading the signal in a time dependent manner from a selected pixels on a multipixel detector such as a photo diode array; (ii) interpreting the read signal with those algorithms to filter noise, compute wavelength or frequency value from the input signal, perform a quality assessment of the computed values; and (iii) producing an output value for further analysis. The further analysis may comprise human interpretation or additional computational processing.

In many cases, the output is an electropherogram showing the detected signals as peaks for identification and analysis. As described above, an electropherogram is a plot of results recording the separated components of a biological sample produced by capillary electrophoresis (see FIG. 5 and Examples 2-5). The electropherogram may comprise several peaks, each corresponding to the relative molar concentration and/or particle number of a fluorophore-labeled lipoprotein component in the biological sample (see Examples 2-5). The total area under each peak corresponds to the total signal detected in a sample.

Some dimension or representation of a signal's peak may be proportional to the molar concentration of the apolipoprotein or lipoprotein particle of interest. The output value may also be indicative of the risk of developing a cardiovascular or metabolic disorder.

Signal processing may be accomplished using various technologies known in the art. An exemplary technology for use in signal processing according to the present invention is deltaDOT's multipixel detection technology. A unique property of deltaDOT's multipixel detection technology is that it allows the tracking of each analyte peak as it moves across the capillary viewing region. By taking multiple images of the analyte at different spatial positions a direct measurement of the velocity of each peak as it traverses the 512 pixel photo diode array may be obtained.

The tracking concept and general principle is illustrated in FIGS. 5A-5C. The analysis consists of three stages. First, peak searching is performed on each individual pixels electropherogram. Each peak detected is quantified in terms of migration time and peak area (or peak height). Next the algorithm sorts through all of the peaks and tries to assign them to tracks, which represents the path of the analytes across the capillary window. Once a set of peaks has been assigned to a track, a linear fit is used to determine the velocity of the analyte averaged across all of the pixels.

The system may further comprise a storage module for the output value thus obtained. Further, the system comprises a module for generating a report based on output value for the user.

The report may include, among other things, the molar concentration and/or particle number of a fluorophore-labeled apolipoprotein and/or lipoprotein in a biological sample; an output value indicative of the risk of developing a cardiovascular disease or metabolic disorder; and a description of a recommended treatment regimen based on a cardiovascular disease or metabolic disorder risk assessment.

In some embodiments, the results of lipoprotein analyses are reported in such a report. A report refers in the context of lipoprotein and other lipid analyses to a report provided, for example to a patient, a clinician, other health care provider, epidemiologist, and the like, which includes the results of analysis of a biological specimen, for example a plasma specimen, from an individual. Reports can be presented in printed or electronic form, or in any form convenient for analysis, review and/or archiving of the data therein, as known in the art.

A report may include identifying information about the individual subject of the report, including without limitation name, address, gender, identification information (e.g., social security number, insurance numbers), and the like.

A report may include biochemical characterization of the lipids in the sample in addition to Lp(a), for example without limitation triglycerides, total cholesterol, LDL cholesterol, and/or HDL cholesterol, and the like.

The term “reference range” and like terms refer to concentrations of components of biological samples known in the art to reflect typical normal observed ranges in a population of individuals. A report may further include characterization of lipoproteins, and reference ranges therefore, conducted on samples prepared by the methods provided herein.

Exemplary characterization of lipoproteins in an analysis report may include the concentration and reference range for VLDL, IDL, Lp(a), LDL and HDL, and subclasses thereof. A report may further include lipoprotein size distribution trends.

### EXAMPLES

The following examples are provided to illustrate embodiments of the present invention but they are by no means intended to limit its scope.

### Example 1

### Optical Apparatus for use in CE-ITP-LIF Systems

A schematic of an optical apparatus comprising two optical zones for use in a CE-ITP-LIF system is shown in FIG. 1. Optics zone 1 comprises an optical rail on which are arranged a 445 nm or other specific wavelength laser or laser diode. Light from these sources is focused through a series of optical components comprising, but not limited to, a line generator, a crossed linear polarizer, and a neutral density filter. Light from optics zone 1 is focused onto a 12.5 mm area of a 100 μM internal diameter fused silica capillary (˜365 μM o.d.) in which a 20 mm viewing window has been created by thermal removal of the polyamide sheath. The light then passes through the sample that is being separated by ITP and excites the fluorescent label attached to each analyte molecule (e.g., a lipoprotein and/or lipid particle). Emitted light energy, at a wavelength specific to the fluorescent label is then focused onto a 512 pixel photo diode array (“PDA”) through another series of optical components in optics zone 2. Optics zone 2 comprises a set of imaging lenses (e.g., convex lenses), and an orthogonal crossed linear polarizer. After passing through a cut-on filter that transmits above a certain wavelength, the light energy reaches the detector where the data is acquired on the PDA and the signal is processed by signal processing algorithms.

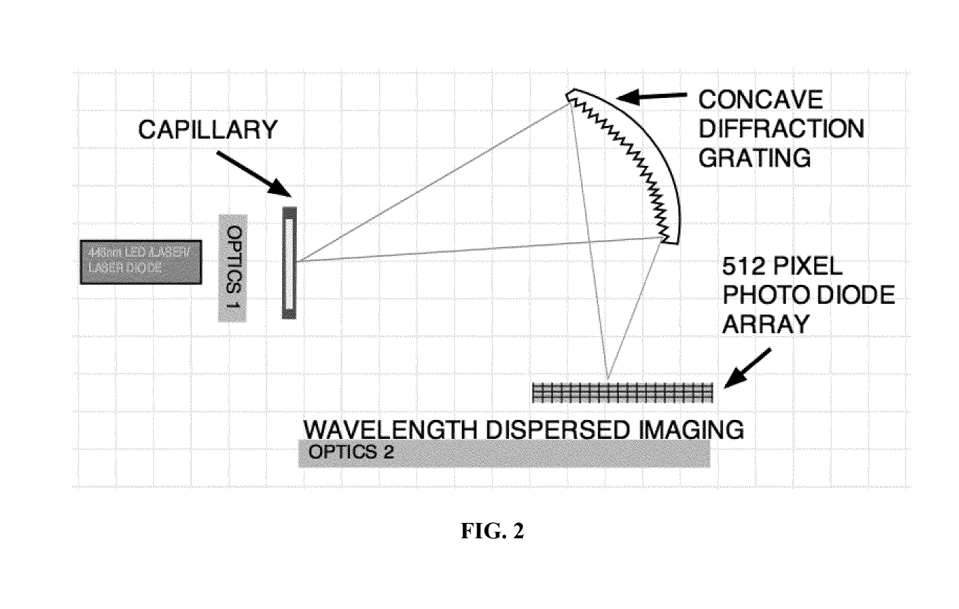

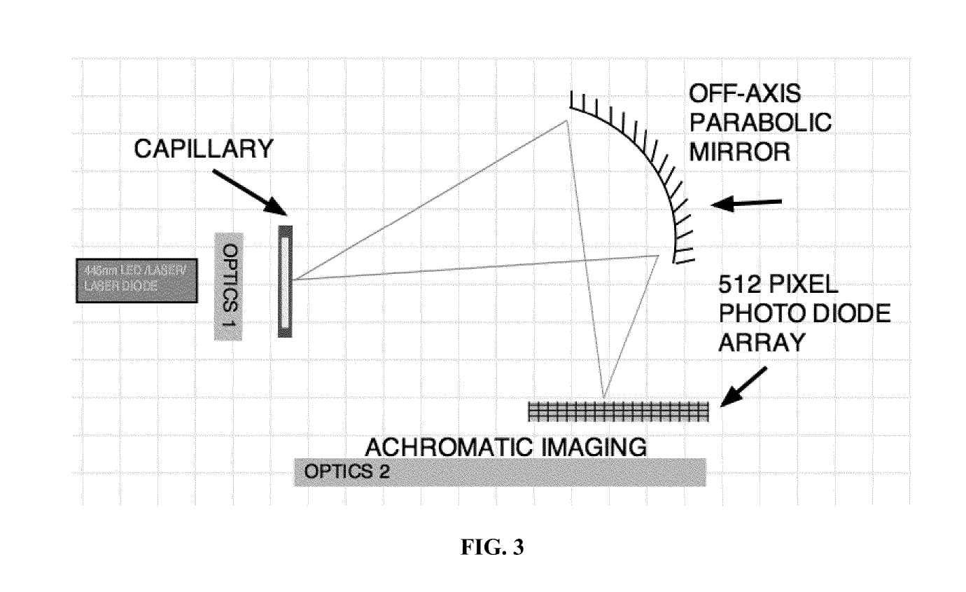

FIG. 2 shows an optical apparatus with a 445 nm LED/Laser/Laser Diode in optics zone 1 and an off axis concave diffusion grating in optics zone 2. The diffusion grating focusses wavelength dispersed achromatic light of a wavelength specific to the fluorescent label onto the 512 pixel photo diode array. By rotating the diffusion grating, the light energy reaches the detector where the data is acquired on the PDA and the signal is processed by proprietary signal processing algorithms. An additional cut-on filter or crossed polarizer may be added. A simple off axis parabolic mirror may replace the diffusion grating (FIG. 3).

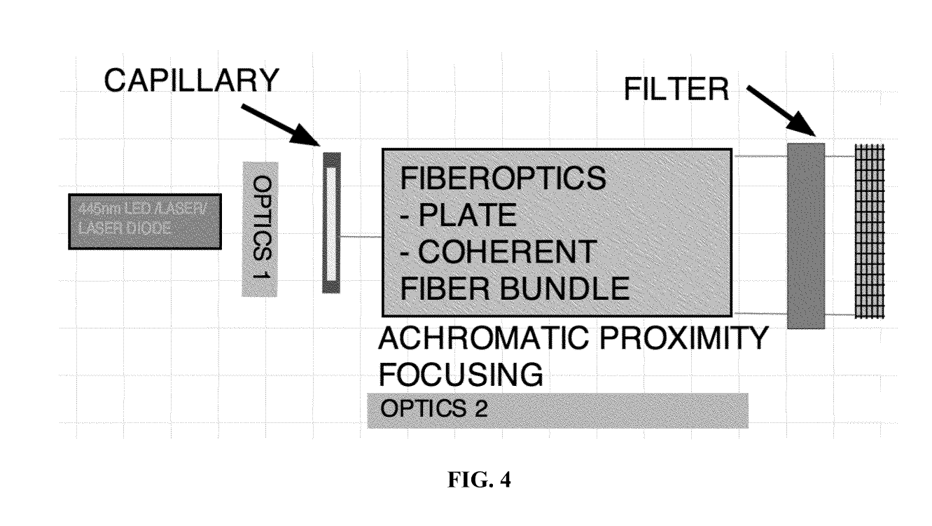

FIG. 4 is a schematic of an optical system comprising a fibre-optic plate (“FOP”) or coherent fibre bundle in optics zone 2. This configuration allows for proximity focusing via a cut-on filter without needing the PDA to touch the capillary (FIG. 4).

### Materials and Methods for Examples 2-5

Leading and Terminating Electrolytes.

The leading electrolyte consists of 10 mm HCL, 0.3% w/v hydroxypropylmethylcellulose (“HPMC”), and 17 mM 2-amino-2-methyl-1,3-propanediol (“Ammediol”). The terminating electrolyte contained 20 mM alanine, 17 mM Ammediol, and was adjusted to pH 10.6 with saturated barium hydroxide solution.

Preparation of Spacer Solutions.

Spacer solutions were prepared to a concentration of 0.32 mg/ml in deionized water and stored at 4° C. Various spacers were made from stock solutions of the following compounds: N-2-acetamido-2-aminoethanesulfonic acid (“ACES”), D-glucuronic acid, octane-sulfonic acid, 2-[(2-Hydroxy-1,1-bis(hydroxymethyl) ethyl)amino]ethanesulfonic acid, 3-[[1,3-dihydroxy-2-(hydroxymethyl)propan-2-yl]amino]propane-1-sulfonic acid, serine, glutamine; methionine, and glycine.

Preparation of the Internal Standard.

1 mg/ml and 2.8 mg·ml carboxyfluorescein (“CF”) solution was prepared in deionized water (“DI”) water and isolated from light.

Biological Samples.

Biological samples were prepared from patients identified as 1, 2, 3, 4, 5, 6, 7, and 8. Patient samples 1, 2, and 6 were previously identified as Lp(a) positive. Patient sample 4 was previously identified as negative for Lp(a).

Biological Sample Preparation.

Biological samples comprising lipoproteins were stained with the fluorescent lipophilic dye 7-nitro-benz-2-oxa-1,3-diazole (“NBD”) ceramide. Briefly, 5 μl of a biological sample were diluted in 37.5 μl deionized water. The diluted sample was incubated for 1 minute with 20 μl NBD-ceramide solution (0.5 mg/ml in ethylene glycol:DMSO, 9:1 (v/v)), mixed with 100 μl of spacer solution (0.32 mg/ml), and spiked with 2.5 μl of the carboxyfluorescein internal standard. In some instances, NBD-ceramide was omitted and replaced with 20 μl of DI water. For biological samples evaluated in the presence of a lipoprotein spike, 2.5 μl of the biological sample was combined with 2.5 μl of the lipoprotein spike prior to dilution in deionized water.

Sample Loading and Data Acquisition.

Samples were injected into a 20 cm Rxi capillary (100 μm) using pressurized injection for 9 seconds at 1 psi. Separation was performed at constant 8 kV. The separated zones were monitored with laser-induced fluorescence detection (excitation 445 nm; emission 550 nm).

Data Analysis and Signal Processing.

Data analysis consists of three stages. First, peak searching is performed on each individual pixel electropherogram (FIG. 5A). Each detected peak is quantified in terms of migration time and peak area (or peak height). Peak area correlates to the particle number of a detected analyte. Next, an algorithm sorts through all of the detected peaks and assigns them to tracks, which represent the path of the analytes across the capillary window (FIG. 5B). Once a set of peaks has been assigned to a track, a linear fit is used to determine the velocity of the analyte averaged across all of the pixels (FIG. 5C), which is needed for signal averaging between pixels.

### Example 2

### Replicate Lipoprotein Profiles of a Single Biological Sample

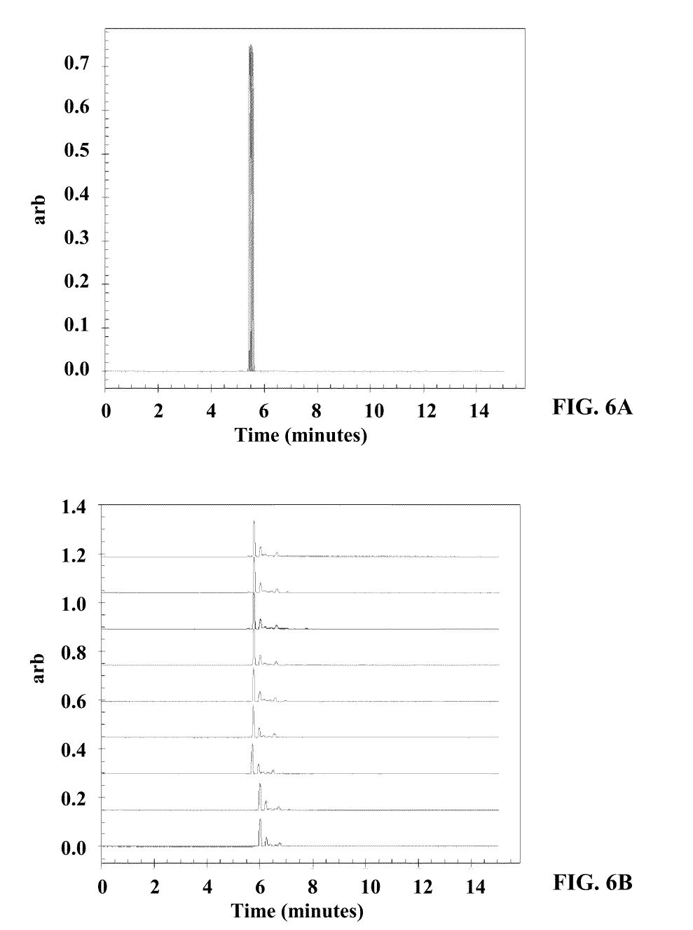

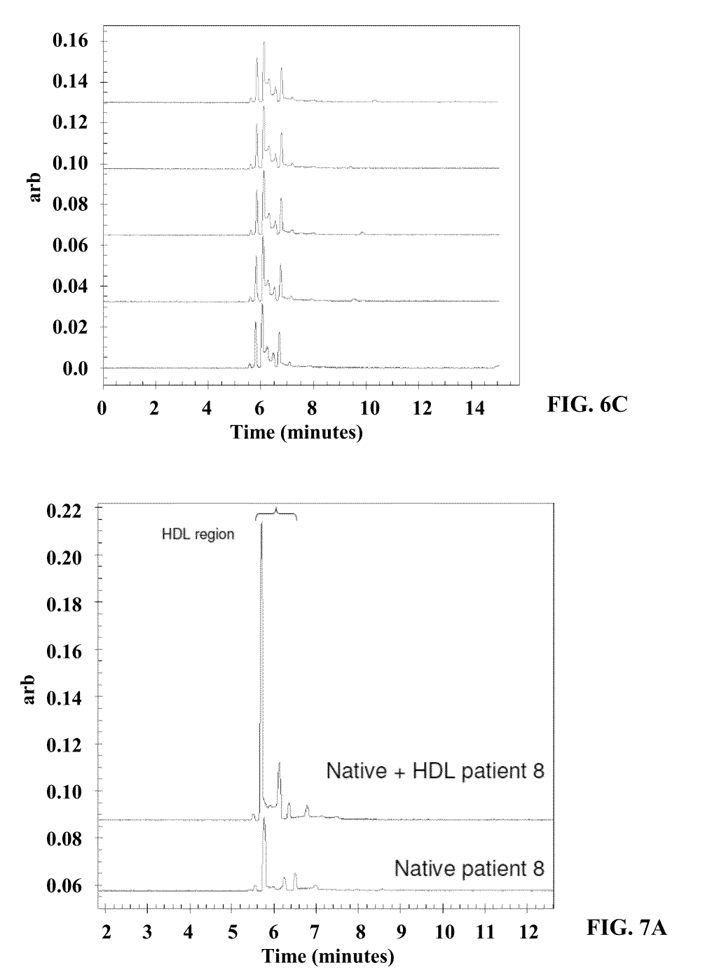

To test the reproducibility of the CE-ITP-LIF system, several replicate biological samples from a single patient were evaluated. As a control experiment, the non-specific lipophilic dye CF was run on the ITP system in the absence of a biological sample. FIG. 6A shows an electropherogram of the control experiment with a peak corresponding to CF (migration time=0.7999), area under peak=2.345). Next, lipoprotein particles in replicate biological samples from patient 8 were labeled with CF and run with a standard CF sample. FIG. 6B is an electropherogram showing the lipoprotein profile of each replicate sample tested. The lipid profile remains constant even after CF has degraded (FIG. 6C).

### Example 3

### Lipoprotein Particle Spiking Results in a Marked Increase in the Corresponding Detected Lipoprotein Peak Height

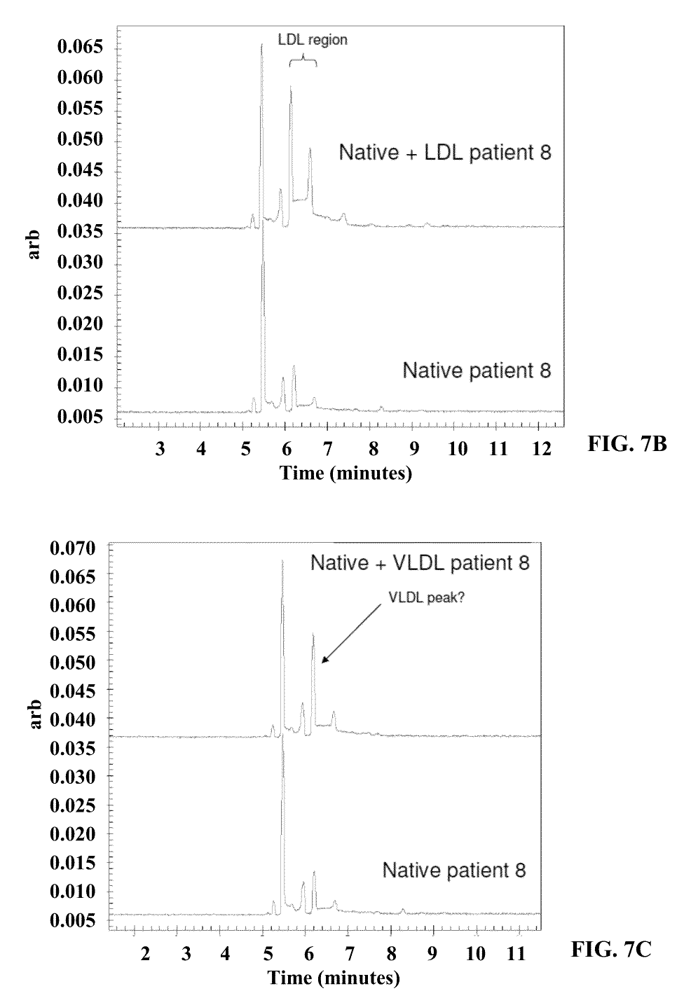

The lipoprotein profile of a biological sample stained with NBD-ceramide generates several peaks corresponding to individual serum lipoproteins (FIGS. 6A-6B). To validate the identity of each individual lipoprotein peak, biological samples were spiked with known amounts of purified lipoprotein. To validate peaks corresponding to HDL and LDL, native samples from patient 8 were spiked with purified HDL and LDL, respectively. The lipid profile of the HDL spiked sample (FIG. 7A, top) and the LDL spiked sample (FIG. 7B, top) were aligned with the lipid profile generated by the native sample (FIG. 7A, bottom; FIG. 7, bottom). As shown in FIG. 7A, there was a marked increase in the peak height and area under the peak in the HDL spiked sample compared to the native sample. FIG. 7B shows the same relationship between the LDL spiked sample compared to the native sample. FIG. 7C shows the lipid profile of a VLDL spiked sample compared to a native sample from patient 8. The VLDL peak (FIG. 7, arrow) seems to fall within the region identified by the LDL spiked sample in FIG. 7B.

### Example 4

### Evaluation of Multiple Biological Samples

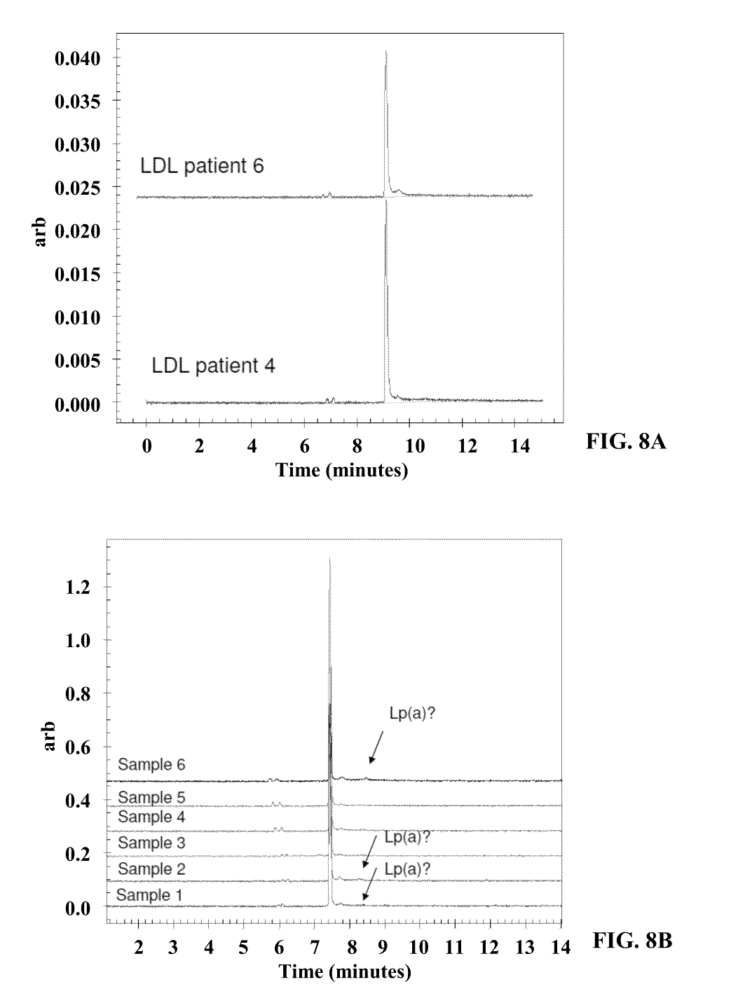

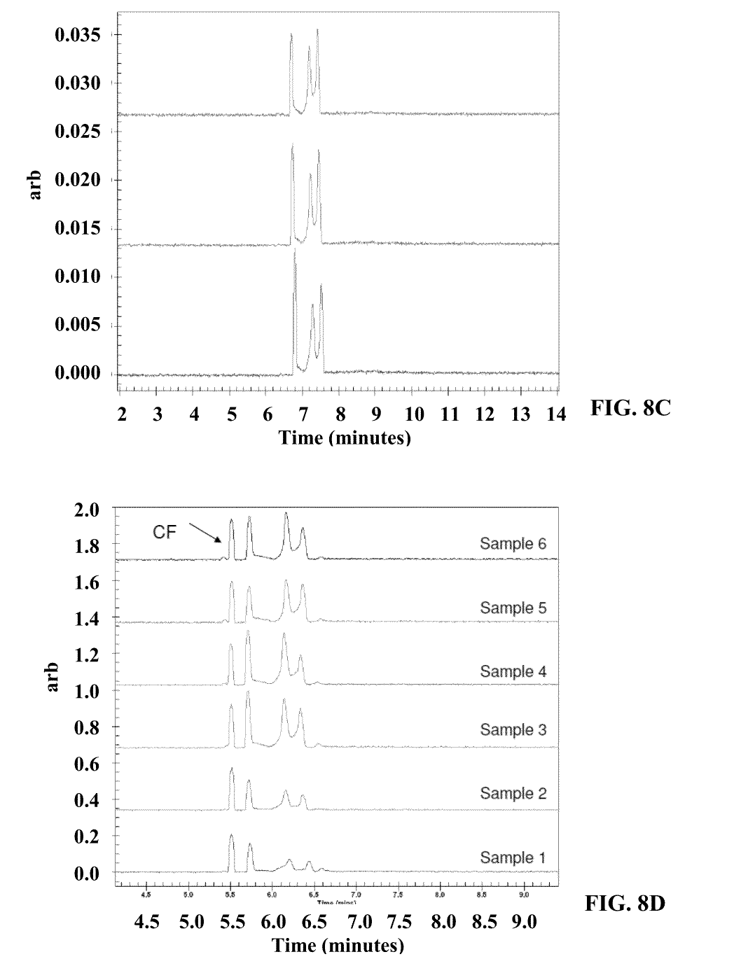

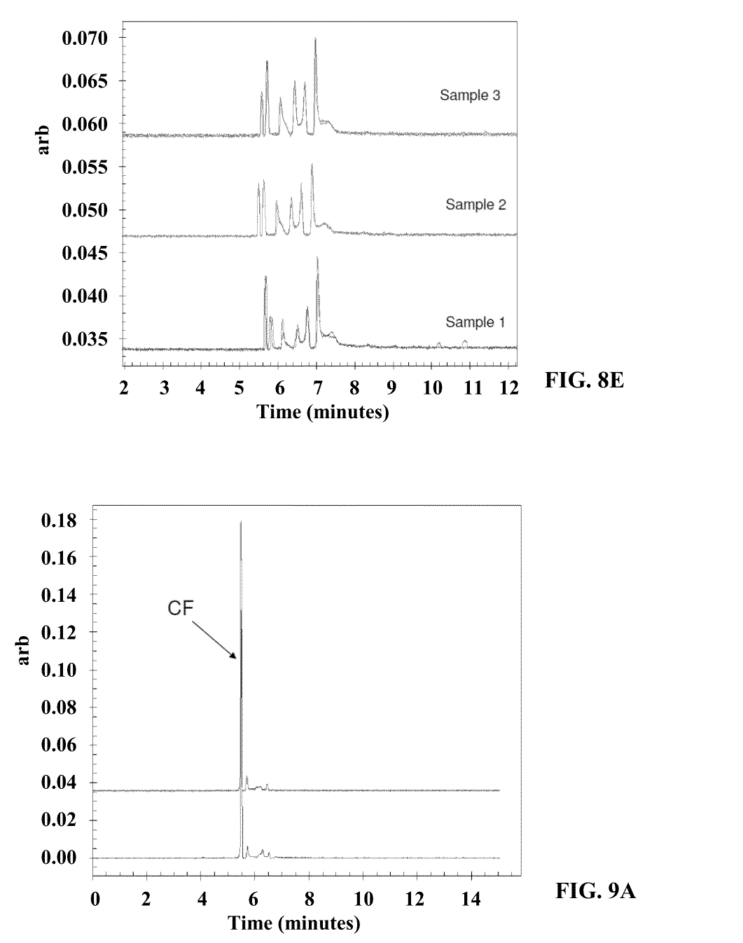

To further evaluate the reproducibility of the system, several samples with known lipoprotein profiles were evaluated. Samples from patients 1, 2, and 6 were previously determined to be Lp(a) positive. Samples from patient 4 were previously determined to be Lp(a) negative. FIG. 8A shows an alignment of the lipid profiles from patient 6 (top) and patient 4 (bottom). The arrows in FIG. 8B indicate the possible location of a Lp(a) peak in samples 1, 2, and 6.

### Example 5

### Quantification of HDL and LDL

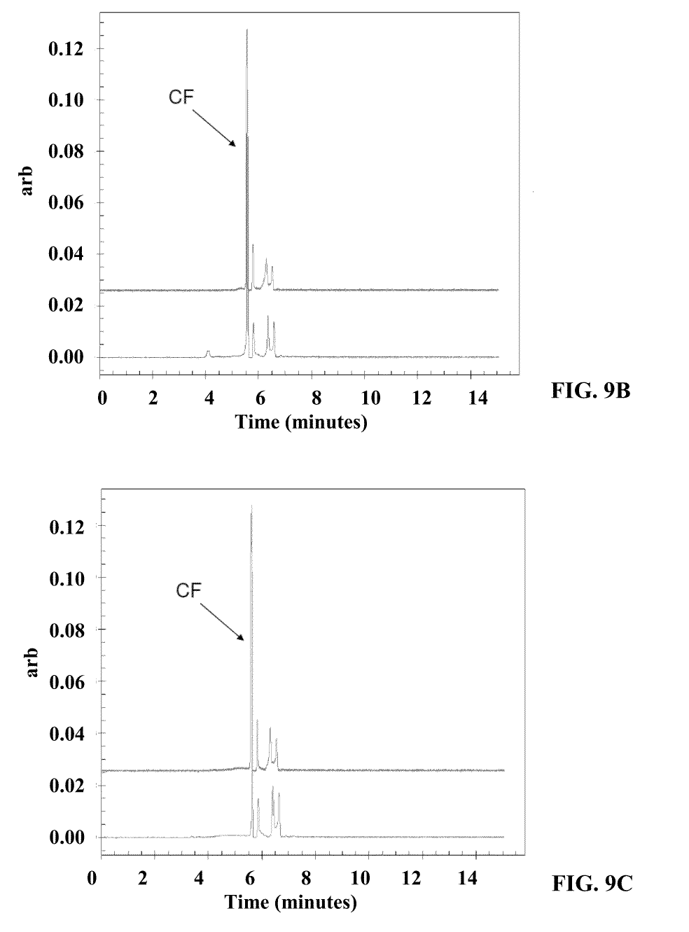

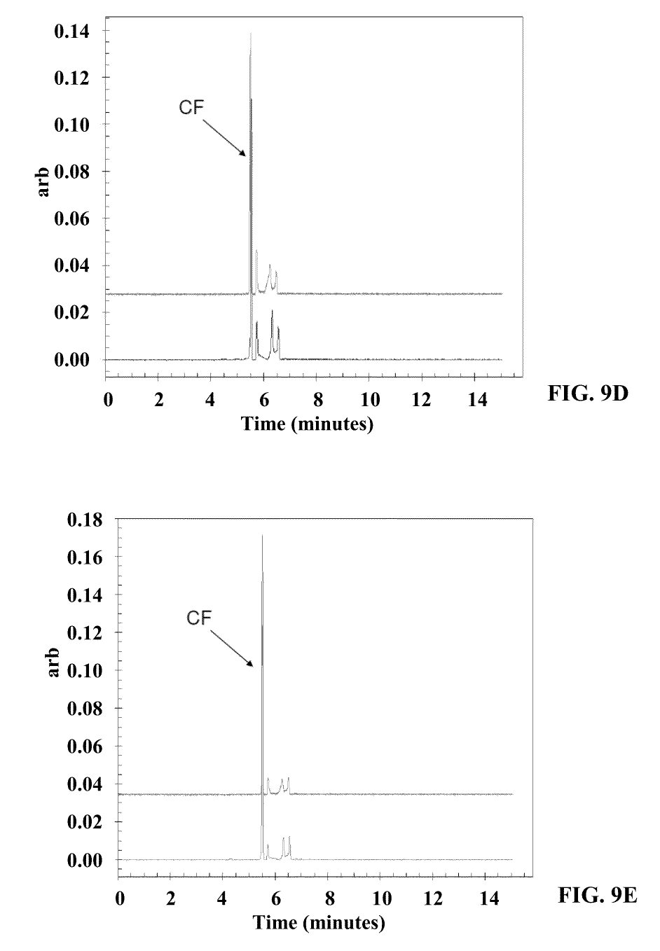

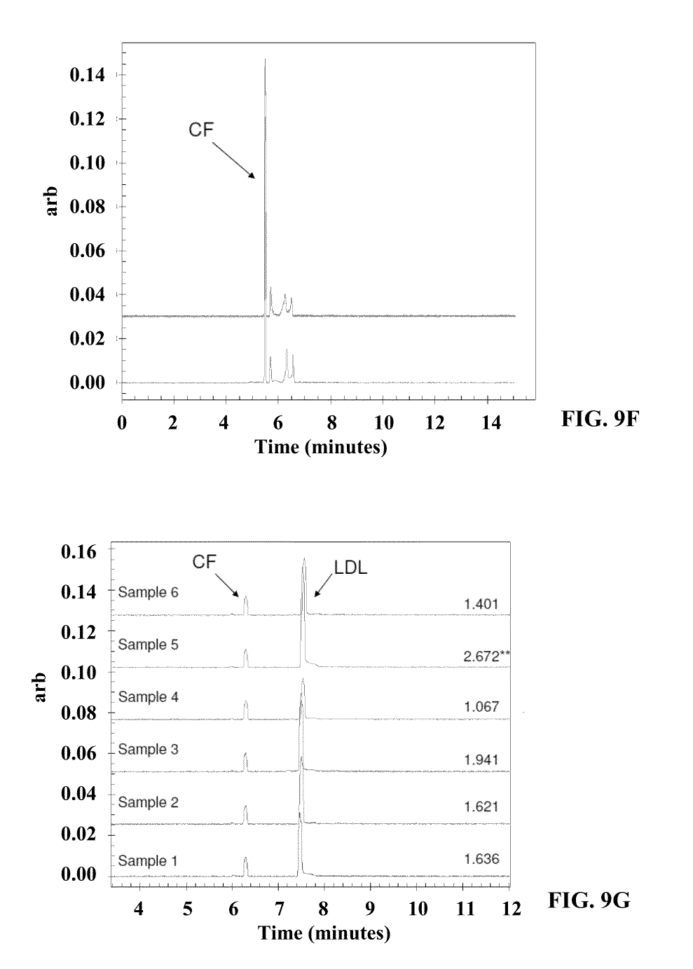

To determine the amounts of HDL and LDL in each of the six patient samples, samples were compared and relative quantities were calculated. Individual electropherograms corresponding to samples 1-6 are shown in FIGS. 9A-9F. The electropherograms were aligned and normalized around the CF peak, which accounts for any fluctuations in the injection (FIG. 9G). The relative amount of HDL in each of the 6 patient samples is shown in Table 1 below. It is possible that sample 4 in FIG. 9G may have had a 2×CF spike. Accordingly, the corrected area would be half of that indicated on the graph for this sample (FIG. 9D).

TABLE 1

| | | Corrected Peak | |
|---|---|---|---|
| Sample | Peak | Area | % Area |
| HDL 1 | CF | 0.764 | 83.15 |
| | HDL A | 0.0638 | 6.95 |
| | HDL B | 0.0593 | 6.46 |
| | HDL C | 0.0224 | 2.44 |
| | HDL D | 0.00926 | 1.01 |
| | Total HDL | 0.16 | |
| HDL 2 | CF | 0.614 | 64.3 |
| | HDL A | 0.112 | 11.69 |
| | HDL B | 0.128 | 13.44 |
| | HDL C | 0.0953 | 9.98 |
| | HDL D | 0.00575 | 0.6 |
| | Total HDL | 0.34 | |
| HDL 3 | CF | 0.546 | 57.91 |
| | HDL A | 0.121 | 12.8 |
| | HDL B | 0.14 | 14.81 |
| | HDL C | 0.131 | 13.85 |
| | HDL D | 0.0059 | 0.63 |
| | Total HDL | 0.4 | |
| HDL 4 | CF | 0.623 | 59.43 |
| | HDL A | 0.136 | 12.99 |
| | HDL B | 0.171 | 16.26 |
| | HDL C | 0.113 | 10.75 |
| | HDL D | 0.0059 | 0.56 |
| | Total HDL | 0.43 | |
| HDL 5 | CF | 0.671 | 72.23 |
| | HDL A | 0.0716 | 7.71 |
| | HDL B | 0.0885 | 9.53 |
| | HDL C | 0.0927 | 9.98 |
| | HDL D | 0.00517 | 0.56 |
| | Total HDL | 0.26 | |
| HDL 6 | CF | 0.645 | 66.16 |
| | HDL A | 0.0989 | 10.14 |
| | HDL B | 0.131 | 13.47 |
| | HDL C | 0.0959 | 9.83 |
| | HDL D | 0.00392 | 0.4 |
| | Total HDL | 0.33 | |

What is claimed is:

1. A method for determining the molar concentration and/or particle number of a lipoprotein in a biological sample, comprising:
(a) contacting the biological sample with a fluorophore-labeled antibody under conditions suitable for the fluorophore-labeled antibody to bind to the lipoprotein, or an immunologically active component thereof, to form a fluorophore-labeled lipoprotein;
(b) subjecting the fluorophore-labeled lipoprotein to a capillary isotachophoresis laser-induced fluorescence (CE-ITP-LIF) system;
(c) detecting signals produced by the fluorophore-labeled lipoprotein; and
(d) quantifying, based on said detecting, the molar concentration and/or particle number of the lipoprotein in the sample, wherein the detected signals are proportional to the molar concentration and/or particle number of the lipoprotein in the sample.

2. The method of claim 1, wherein the immunologically active component of the lipoprotein comprises an apolipoprotein or a domain thereof.

3. The method of claim 1, wherein the CE-ITP-LIF system separates the components of the sample from one another along a common capillary.

4. The method of claim 1, wherein the lipoprotein is an apolipoprotein-containing lipoprotein selected from the group consisting of very low-density lipoprotein (VLDL), low-density lipoprotein (LDL), intermediate-density lipoprotein (IDL), high-density lipoprotein (HDL), chylomicron, lipoprotein X, lipoprotein(a), and subforms and mixtures thereof.

5. The method of claim 4, wherein the apolipoprotein is selected from the group consisting of apolipoprotein A, apolipoprotein B, apolipoprotein C, apolipoprotein D, apolipoprotein E, apolipoprotein H, and oxidized variants and mixtures thereof.

6. The method of claim 1, wherein the CE-ITP-LIF system is a multiplex capillary isotachophoresis laser induced fluorescence (MPCE-ITP-LIF) system.

7. The method according to any of the preceding claims, wherein the sample further comprises a signal-producing calibrator lipoprotein comprising a standard lipoprotein with a known concentration, a known particle number, a known apolipoprotein, a known apolipoprotein concentration, a known apolipoprotein domain, or a combination thereof.

8. The method of claim 7, wherein the signal produced by the signal-producing calibrator lipoprotein is measured and compared with the signal produced from the fluorophore-labeled lipoprotein and the molar concentration and/or particle number of the lipoprotein is determined based on said comparison.

9. The method of claim 1, wherein the fluorophore-labeled antibody is an intact antibody or antibody fragment.

10. The method of claim 7, wherein the signals produced from the fluorophore-labeled antibody bound to the lipoprotein, or to the immunologically active component thereof, are proportional to the molar concentration and/or particle number of the lipoprotein in the sample.

11. A method of assessing cardiovascular risk in a subject, comprising:
(i) determining the particle number and/or molar concentration of a lipoprotein in a biological sample from the subject; and
(ii) assessing the cardiovascular risk of the subject based on the particle number and/or molar concentration of the lipoprotein; wherein the particle number and/or molar concentration of the lipoprotein is determined by:
(a) contacting the biological sample with a fluorophore-labeled antibody under conditions suitable for the fluorophore-labeled antibody to bind to the lipoprotein or an immunologically active component thereof, to form a fluorophore-labeled lipoprotein;
(b) subjecting the fluorophore-labeled lipoprotein to a capillary isotachophoresis laser-induced fluorescence (CE-ITP-LIF) system;
(c) detecting signals produced by the fluorophore-labeled lipoprotein; and
(d) quantifying, based on said detecting, the particle number and/or molar concentration of the of the lipoprotein in the sample, wherein the detected signals are proportional to the particle number and/or molar concentration of the lipoprotein.

12. The method of claim 11, wherein the CE-ITP-LIF system separates the components of the sample from one another along the common capillary.

13. The method of claim 11, wherein the sample further comprises a signal-producing calibrator lipoprotein comprising a standard lipoprotein with a known concentration, a known particle number, a known apolipoprotein, a known apolipoprotein concentration, a known apolipoprotein domain, or a combination thereof.

14. The method of claim 13, wherein the signal produced by the signal-producing calibrator lipoprotein is measured and compared with the signal produced from the fluorophore-labeled lipoprotein and the molar concentration and/or particle number of the lipoprotein is determined based on said comparison.

15. The method of claim 11, wherein the signals from the fluorophore-labeled antibody bound to the lipoprotein, or to the immunologically active component thereof, are proportional to the molar concentration and/or particle number of the lipoprotein in the sample.

16. The method of claim 11, wherein the subject is assigned to one of a low, moderate, or high cardiovascular risk categories based on the particle number and/or molar concentration of the lipoprotein.

17. The method of claim 16, wherein the method further comprises administering to the subject a therapeutic regimen for reducing the cardiovascular risk, or modifying an existing therapeutic regimen for the subject for reducing the cardiovascular risk, based on the cardiovascular risk category assigned to the subject.

18. The method of claim 17, wherein the therapeutic regimen comprises administering a drug and/or a supplement or the existing therapeutic regimen comprises administering a modified dose of a drug and/or a supplement.

19. The method of claim 18, wherein the drug is selected from the group consisting of niacin, an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, an angiotensin system inhibitor, and combinations thereof.

20. The method of claim 18, wherein the drug is selected from the group consisting of niacin, statin, ezetimibe, fenofibrate, estrogen, raloxifene and any combinations thereof.

21. The method of claim 17, wherein the selected therapeutic regimen involves giving recommendations on making or maintaining lifestyle choices based on the results of said cardiovascular risk determination.

22. The method of claim 21, wherein the lifestyle choices involve changes in diet, changes in exercise, reducing or eliminating smoking, or a combination thereof.

23. The method according to any of the preceding claims, wherein the biological sample is selected from the group consisting of blood, plasma, urine and saliva.

24. The method according to any of the preceding claims, wherein the signal produced by the fluorophore-labeled antibody is measured and the molar concentration and/or particle number of the lipoprotein is determined based on the following formula:
PN=(relative AUC value)×(total apolipoprotein concentration)
wherein:
PN is equal to particle number in
$$( \frac{nmol}{L} ) ;$$
(relative AUC value) is determined by calculating the relative optical density of a specific peak as a portion of the optical density of all peaks; and (total apolipoprotein concentration) is given in
$$( \frac{nmol}{L} )$$
and is determined by measuring the specific total apolipoprotein concentration.

25. The method of claim 24, wherein the method further comprises
(a) calculating a score of measured levels of apolipoprotein by converting the apolipoprotein concentration to molar lipid concentration;
(b) comparing the score to a score obtained from a population of patients; and
(c) assigning a cardiovascular risk level to the subject, based on said comparing.

26. A system for determining the molar concentration and/or particle number of a lipoprotein in a biological sample, comprising:
a capillary electrophoresis apparatus for separating components of a moiety-bound sample, wherein the moiety-bound sample is prepared by contacting the biological sample with a fluorophore-labeled antibody under conditions suitable for the fluorophore-labeled antibody to bind to the lipoprotein or an immunologically active component thereof, to form a fluorophore-labeled lipoprotein;
a detector for detecting signals produced by the fluorophore-labeled lipoprotein; and
a processor for quantifying, based on said detecting, the concentration and/or particle number of the lipoprotein in the sample, wherein the detected signals are proportional to the molar concentration and/or particle number of the lipoprotein in the sample.

27. The system of claim 26, wherein the system is a capillary isotachophoresis laser-induced fluorescence (CE-ITP-LIF) system.

28. The system of claim 27, wherein the system is a multiplex capillary isotachophoresis laser induced fluorescence (MPCE-ITP-LIF) system.

29. The method of claim 24, wherein the total apolipoprotein concentration was measured by polyacrylamide gel electrophoresis.
